(19) United States
(12) Patent Application Publication
(10) Pub. No.: US 2024/0399047 A1
(43) Pub. Date: Dec. 5, 2024

(54) INTERMITTENT SUCTION REGULATION DEVICE AND METHOD

(71) Applicant: Zimmer, Inc., Warsaw, IN (US)

(72) Inventors: Maxim Budyansky, Warsaw, IN (US); Marton Varady, Warsaw, IN (US); Evan Fair, Warsaw, IN (US); George Andrews, Warsaw, IN (US); Neil Shah, Warsaw, IN (US)

(21) Appl. No.: 18/662,691

(22) Filed: May 13, 2024

Related U.S. Application Data
(60) Provisional application No. 63/470,043, filed on May 31, 2023.

(51) Int. Cl. A61M 1/00; A61M 39/22

(52) U.S. Cl. CPC A61M 1/75; A61M 1/743; A61M 39/223; A61M 2039/226; A61M 2205/3337; A61M 2205/50

(57) ABSTRACT

Methods and devices for regulating suction pressure are disclosed. The suction regulator device may include a housing including three ports and a valve engaging the ports. The method includes connecting a vacuum source to the first port, connecting a suction powered device to the third port, and oscillating the valve between maintaining a closed configuration for a predetermined time period and maintaining an open configuration for a second predetermined time period. In the closed configuration the valve places the third port in fluid communication with the second port exposing to ambient/atmospheric pressure and in the open configuration the valve places the third port in fluid communication with the first port.

### CLAIM OF PRIORITY

This application claims the benefit of U.S. Provisional Patent Application Ser. No. 63/470,043, filed on May 31, 2023, the benefit of priority of which is claimed hereby, and which is incorporated by reference herein in its entirety.

### SUMMARY

Methods and devices for regulating vacuum suction in medical/surgical applications are disclosed.

### BACKGROUND

Vacuum suction is applied in various orthopaedic surgical procedures, including surgical procedures that require the evacuation of bone or marrow material, such as bone or marrow harvesting. There are orthopaedic needs to remove liquid from the area of removal while reducing the coagulative stresses due to excess suction. Currently, the regulation of suction is inefficient at preventing coagulation of peripheral blood, resulting in suboptimal harvesting of bone or marrow material in the presence of peripheral blood.

### BRIEF DESCRIPTION OF THE DRAWINGS

FIGS. 1A-1D schematically show views of a suction regulator device according to the present disclosure.

FIG. 2 schematically shows an exploded view of the device of FIG. 1.

FIGS. 3A and 3B schematically show ports of a suction regulator device in a closed configuration and an open configuration according to the present disclosure.

FIG. 3C schematically shows a check valve implemented in a suction regulator device according to the present disclosure.

FIG. 4 is a graph depicting a harvest score associated with various methods of operating a suction regulator device.

### DETAILED DESCRIPTION

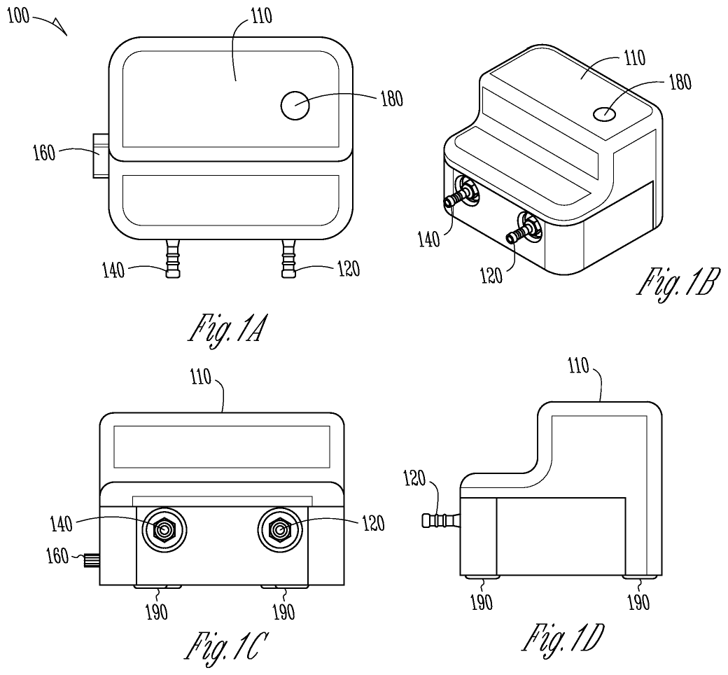

FIG. 1A schematically shows a top view of a suction regulator device 100. FIG. 1B schematically shows a perspective view of a suction regulator device 100. FIG. 1C schematically shows a front view of a suction regulator device 100. FIG. 1D schematically shows a side view of a suction regulator device 100.

The device 100 includes a housing 110. Situated relative to a surface of the housing 110 is a first port 120, a second port 130, and a third port 140. In some embodiments, a plurality of the ports may be situated within or relative to the same surface of the housing 110. In other embodiments, each port is situated within or relative to a different surface of the housing 110.

First port 120 is configured to connect to a vacuum source outside of the housing 110. The vacuum source may be any conventional vacuum source known to those of skill in the art. Second port 130 is configured to vent vacuum pressure out of the housing 110 and is located inside the housing. Third port 140 is configured to connect to a suction powered device outside of the housing 110. The suction powered device may be any conventional suction device known to those of skill in the art. In some embodiments, the suction device is capable of evacuating bone and/or marrow material from a harvesting site.

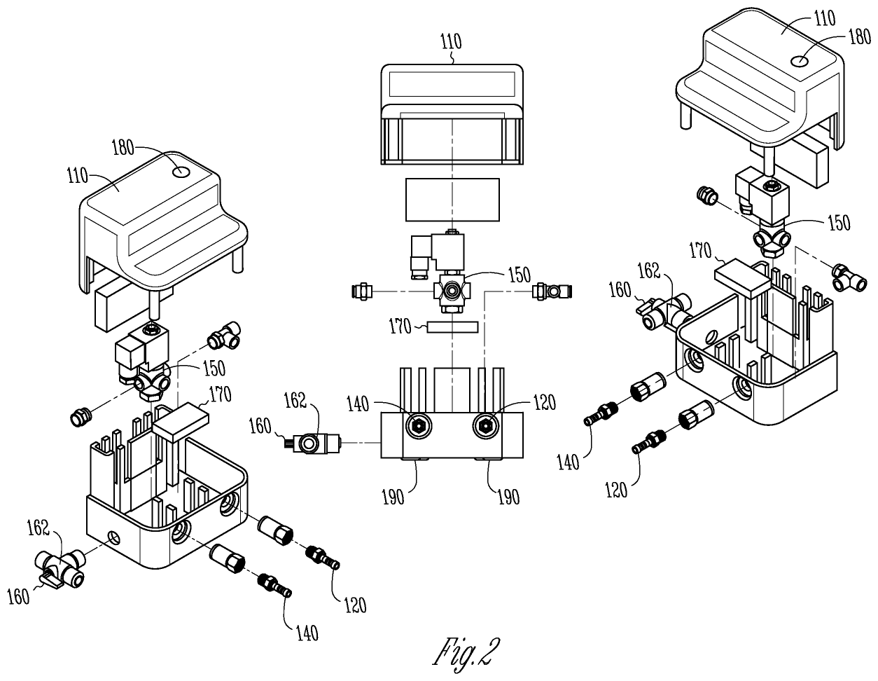

The ports may be engaged in fluid communication with one another by a valve 150. Valve 150 is discussed in further detail below with regards to FIG. 2.

The device 100 further includes a bypass mechanism 160 situated within the surface of the housing 110. In some embodiments, the bypass mechanism 160 is situated on a different surface than any of the ports. In other embodiments, the bypass mechanism 160 is situated on the same surface as one of the ports. The bypass mechanism 160 may be configured to place the first port 120 in fluid communication with the third port 140. In alternate embodiments; the bypass can be configured as an electronic switch that keeps the valve open to apply continuous suction to the connected suction device.

The device 100 further includes an activation mechanism 180 situated within a surface of the housing 110. In some embodiments, the activation mechanism 180 is situated within a top surface of the housing 110. The activation mechanism 180 may initiate operation of the device 100 in a manner as described in further detail below.

In some embodiments, the device 100 further includes one or more supports 190 situated on a bottom surface of the housing 110. The supports 190 are structured to prevent movement of the device 100 when pressure, such as a vacuum pressure, is introduced in the device 100.

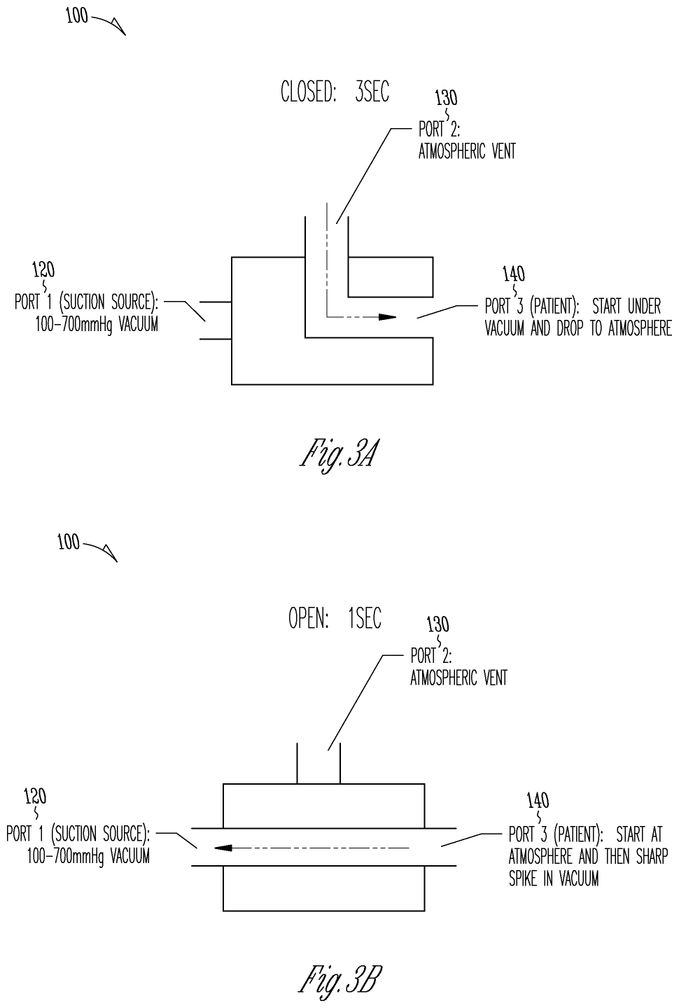

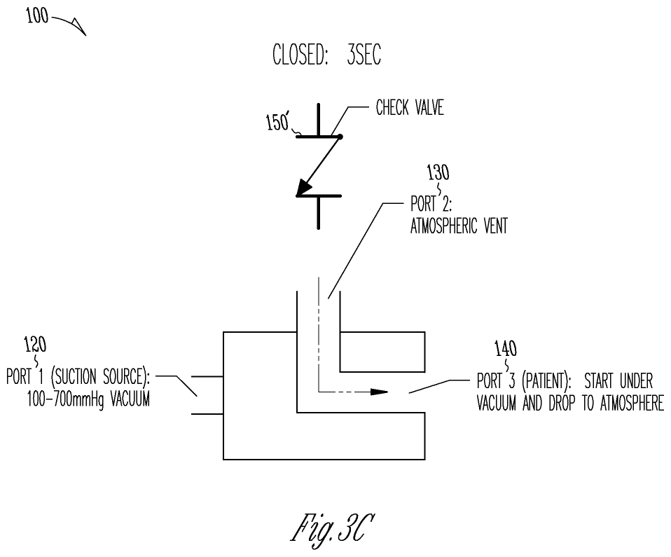

FIG. 2 schematically shows an exploded view of the cartridge of FIG. 1. FIG. 2 depicts the valve 150 that is situated within the housing 110. In some embodiments, the valve 150 is a three-way solenoid valve. Valve 150 is positioned such that it may connect first port 120 in fluid communication with third port 140 or may connect second port 130 in fluid communication with third port 140. When first port 120 is in fluid communication with third port 140, the device 100 is considered to be in an open configuration. When second port 130 is in fluid communication with third port 140, the device 100 is considered to be in a closed configuration. Operation of the valve 150 between a closed configuration and an open configuration is depicted in FIGS. 3A and 3B.

FIGS. 3A and 3B schematically show ports of a suction regulator device 100 in a closed configuration and an open configuration according to the present disclosure. A valve, for example, valve 150, switches between the closed configuration and the open configuration. The valve 150 may be switched on a predetermined interval as determined by a controller 170 situated within or otherwise associated with the housing 110.

In some embodiments, each configuration is maintained for a predetermined period of time. In some embodiments, the closed configuration is maintained for a first predetermined time period ranging from about two seconds to about four seconds. In further embodiments, the first predetermined period of time is about three seconds. In some embodiments, the open configuration is maintained for a second predetermined time period ranging from about two seconds to about half a second. In further embodiments, the second predetermined period of time is about one second. The repeating switching between the closed configuration and open configuration allows for liquid accumulation within the area where suction is applied. When in open-state, liquid and solid materials can be suctioned into a device, line, etc. having access to a suction site. When in closed-state, liquid (peripheral blood) is able to return back to the suction site while the suction equipment is able to keep or maintain at least some of the solid materials that were suctioned.

The controller 170 may be programmable such that the first and second predetermined time periods may be modified.

When in a closed configuration, the suction powered device that is connected to the third port 140 is exposed to atmospheric pressure from the second port 130. In doing so, the suction powered device and associated suction line drop to atmosphere pressure. During this time, the first port 130 is accumulating pressure. In some embodiments, as depicted in FIG. 3C, a check valve 150′ prime is placed at the second port 130 to prevent excess fluid from exiting the ambient facing second port 130.

When in an open configuration, a vacuum pressure is introduced through the first port 120 and into a suction powered device through the third port 140. Upon switching to the open configuration, the suction line is exposed to a sharp spike in vacuum pressure. The vacuum pressure draws material into the suction powered device, for example bone material, marrow material, and associated liquids.

Bypass mechanism 160 may be engaged with a bypass valve 162 situated within the housing 110. In some embodiments, the bypass valve 162 is a three-way ball valve. When engaged by the bypass mechanism 160, the bypass valve 162 maintains the device 100 in the open configuration. In this manner, the bypass mechanism 160 provides continuous suction to the suction powered device. In other embodiments, the bypass mechanism 160 can be an electronic switch instead of a physical bypass. In this embodiment the user can electronically (e.g. use a button or switch) to turn on continuous suction setting.

Device 100 may be powered by one or more batteries. In other embodiments, device 100 may be plugged into a power source.

In certain embodiments, a suction regulator device can comprise a housing with at least a first port, a second port and a third port. The first port can be configured to connect to a vacuum source outside of the housing. The second port can be configured to expose a suction line to ambient pressure. The third port can be configured to connect to a suction powered device outside of the housing via the suction line. The housing can further include a valve that is situated within the housing and can be switchable between an open configuration and a closed configuration. In the open configuration, the third port can be fluidly connected the first port to thereby subject the suction line and the suction powered device to a pressure of the vacuum source. In the closed configuration, the third port can be fluidly connected to the second port to thereby subject the suction line and the suction powered device to ambient pressure. The valve can be controllable for maintaining the open configuration for a first predetermined time period and the closed configuration for a second predetermined time period. The first predetermined time period can be any useful time period, for example, ranging from about one second to about thirty seconds, or about two seconds to about 10 seconds, or about three second to about six seconds, typically from about three seconds to about four seconds. The second predetermined time period can be any useful time period, for example, ranging from about one tenth of a second to about six seconds, or about one quarter of a second to about four seconds, or about half a second to about three seconds, typically from about half a second to about one second. The suction regulator device can be controlled by a controller, for example, one that can switch the valve from the open configuration to the closed configuration and vice versa, for example, from the open configuration to the closed configuration at the end of the first predetermined time period and from the closed configuration to the open configuration at the end of the second predetermined time period. Such switching processes can be repeated any number of times.

A method of regulating suction at a bone harvesting site can include providing a suction regulator device, for example device 100 described above. Before operating the device, a first port of the device is connected to a vacuum source. The vacuum source may be any suitable conventional vacuum source known in the art. Then a suction powered device is connected to the third port of the device. The suction powered device may be any suitable suction device known in the art.

After connecting the device the user may initiate a bone harvesting procedure. The user may engage the device by actuating an activation mechanism on the device. Once activated, the device may oscillate a valve between maintaining a closed configuration for a predetermined time period and maintaining an open configuration for a second predetermined time period as discussed n further detail above. When in a closed configuration, the suction powered device that is connected to the third port is exposed to atmospheric pressure from a second port. When in this closed configuration the suction device connected to the regulator has no active suction at the working end. When in an open configuration, a vacuum pressure is introduced through the first port and into a suction powered device through the third port.

In some embodiments, the device is configured to automatically begin oscillating the valve upon activation. In some embodiments, a controller within the device is programmable to define or revise the predetermined time periods. In some embodiments, the device includes a bypass mechanism. A user may engage the bypass mechanism to keep the device in an open configuration, providing constant suction to the suction powered device.

In some embodiments, the device may be disposed of after completion of a harvesting procedure. In other embodiments, the device may be sterilized for another use.

In certain embodiments, a method of providing discontinuous suction during a surgical procedure can comprise providing or obtaining a suction system that includes a vacuum source, a suction regulator device, and a suction powered device that is fluidly connected to the suction regulator device via a suction line. The suction regulator device can comprise a housing with a first port that is connected to the vacuum source, a second port that exposes the suction line to ambient pressure, and a third port that is connected to the suction powered device via the suction line. In addition, a valve is situated within the housing that is switchable between an open configuration and a closed configuration. In the open configuration, the third port is fluidly connected the first port to thereby subject the suction line and the suction powered device to a pressure of the vacuum source. In the closed configuration, the third port is fluidly connected to the second port to thereby subject the suction line and the suction powered device to ambient pressure. The method then can further comprise delivering the suction powered device to a suction site in a patient, maintaining the valve in the open configuration for a first predetermined time period with the vacuum source activated and the suction powered device at the suction site, and maintaining the valve in the closed configuration for a second predetermined time period with the vacuum source activated and the suction powered device at the suction site.

Operating the device at the discussed predetermined time periods has been found to reduce coagulation stress during the evacuation of bone and marrow. During the closed state, liquid that can coagulate is released from the overall system back to the patient while solid matter stays inside a collection receptacle of the suction powered device.

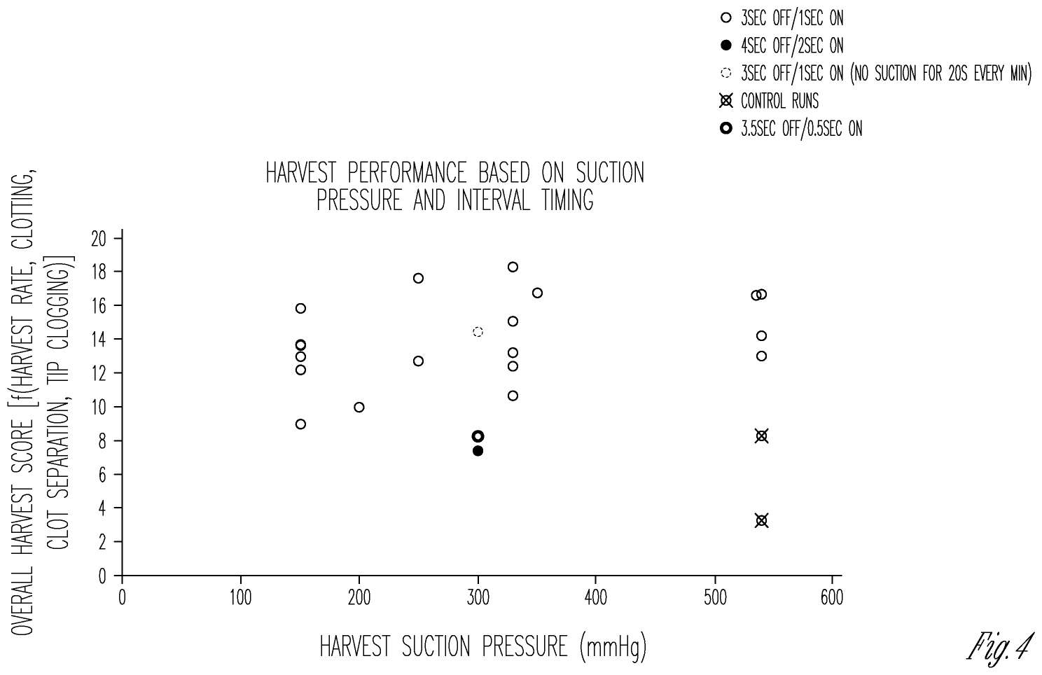

FIG. 4 is a graph depicting a harvest score associated with various methods of operating a suction regulator device. A suction regulator device, similar to device 100 described above, was tested at various pressures and was switched between a closed position and an open position at various predetermined time periods. A harvest score was assigned to each test based on the rate of harvested material, the amount of clotting of the collected contents, clot separation, and clogging of the tip in the suction line.

A control harvest included operation of the device with constant suction. In other words, the device was in a constant open configuration as if the bypass mechanism was engaged. The control harvest was conducted with 540 mmHG of pressure. The control harvest was conducted at two different sites and yielded scores of 3 and 8, approximately.

A harvest was conducted at 300 mmHG where the device maintained a closed configuration for four seconds and an open configuration for two seconds. This harvest obtained a score of 7, approximately.

A harvest was conducted at 300 mmHG where the device maintained a closed configuration for three and a half seconds and an open configuration for half a second. This harvest obtained a score of 8, approximately.

By comparison, harvest scores were appreciably higher at various pressures when the device maintained a closed configuration for three seconds and an open configuration for one second. An additional harvest with these time periods, but including a twenty second pause without suction after one second in an open configuration, obtained a harvest score of approximately 14 at 300 mmHG.

In certain embodiments, a method of providing discontinuous suction during a surgical procedure can comprise providing or obtaining a suction system that includes a vacuum source, a suction regulator device, and a suction powered device that is fluidly connected to the suction regulator device via a suction line. The suction regulator device can comprise a housing with a first port that can be connected to the vacuum source, a second port that can expose the suction line to ambient pressure, and a third port that can be connected to the suction powered device via the suction line. The system can also include a valve that can be situated within the housing and can be switchable, for example, switchable back and forth between an open configuration and a closed configuration. For example, in the open configuration, the third port can be fluidly connected the first port to thereby subject the suction line and the suction powered device to a pressure of the vacuum source, and in the closed configuration, the third port can be fluidly connected to the second port to thereby subject the suction line and the suction powered device to ambient pressure. The method can further include delivering a tip or other part of the suction powered device that includes a suction opening to a suction site in a patient, maintaining the valve in the open configuration for a first predetermined time period with the vacuum source activated and the tip of the suction powered device at the suction site so as to draw both liquid bone matter and solid bone matter into the suction line through the tip, and maintaining the valve in the closed configuration for a second predetermined time period with the vacuum source activated and the tip of the suction powered device at the suction site such that at least some of the liquid bone matter returns to the suction site through the tip and at least some of the solid bone matter remains in the suction line. Such a switching process can be controlled by a controller and can be repeated any number of times during a procedure.

Although the present disclosure has been described with reference to exemplary embodiments and implementations, the present disclosure is not limited by or to such exemplary embodiments/implementations. Rather, the present disclosure is susceptible to revision, modification and/or refinement without departing from the spirit or scope hereof.

What is claimed is:

1. A suction regulator device, comprising:
a housing;
a first port of the housing configured to connect to a vacuum source outside of the housing;
a second port of the housing configured to expose a suction line to ambient pressure;
a third port of the housing configured to connect to a suction powered device outside of the housing via the suction line; and
a valve situated within the housing and switchable between an open configuration and a closed configuration, wherein in the open configuration the third port is fluidly connected the first port to thereby subject the suction line and the suction powered device to a pressure of the vacuum source, and wherein in the closed configuration the third port is fluidly connected to the second port to thereby subject the suction line and the suction powered device to ambient pressure,
wherein the valve is controllable for maintaining the open configuration for a first predetermined time period and the closed configuration for a second predetermined time period.

2. The suction regulator device of claim 1, wherein the valve is a three-way solenoid valve.

3. The suction regulator device of claim 1 further comprising a bypass mechanism for maintaining the valve in the open configuration.

4. The suction regulator device of claim 3, wherein the bypass mechanism comprises a bypass valve.

5. The suction regulator device of claim 4, wherein the bypass valve is an electronic switch.

6. The suction regulator device of claim 1, wherein the first predetermined time period ranges from about two seconds to about four seconds.

7. The suction regulator device of claim 6, wherein the first predetermined time period is about three seconds.

8. The suction regulator device of claim 1, wherein the second predetermined time period ranges from about half a second to about two seconds.

9. The suction regulator device of claim 8, wherein the second predetermined time period is about one second.

10. The suction regulator device of claim 1 further comprising a controller situated within the housing for switching the valve from the open configuration to the closed configuration at the end of the first predetermined time period and from the closed configuration to the open configuration at the end of the second predetermined time period.

11. The regulator of claim 10, wherein the controller is programmable such that the first and second predetermined time periods may each be modified.

12. A method of providing discontinuous suction during a surgical procedure, comprising:
providing or obtaining a suction system that includes a vacuum source, a suction regulator device, and a suction powered device fluidly connected to the suction regulator device via a suction line, the suction regulator device comprising:
a housing;
a first port of the housing connected to the vacuum source;
a second port of the housing exposing the suction line to ambient pressure;
a third port of the housing connected to the suction powered device via the suction line; and
a valve situated within the housing and switchable between an open configuration and a closed configuration, wherein in the open configuration the third port is fluidly connected the first port to thereby subject the suction line and the suction powered device to a pressure of the vacuum source, and wherein in the closed configuration the third port is fluidly connected to the second port to thereby subject the suction line and the suction powered device to ambient pressure;
delivering the suction powered device to a suction site in a patient;
maintaining the valve in the open configuration for a first predetermined time period with the vacuum source activated and the suction powered device at the suction site; and
maintaining the valve in the closed configuration for a second predetermined time period with the vacuum source activated and the suction powered device at the suction site.

13. The method of claim 12 further comprising engaging a bypass valve in the housing to maintain the valve in the open configuration.

14. The method of claim 12, wherein the first predetermined time period ranges from about two seconds to about four seconds.

15. The method of claim 12, wherein the second predetermined time period ranges from about half a second to about two seconds.

16. The method of claim 12, wherein the suction system further comprises a controller situated within the housing for switching the valve from the open configuration to the closed configuration at the end of the first predetermined time period and from the closed configuration to the open configuration at the end of the second predetermined time period.

17. A method of providing discontinuous suction during a surgical procedure, comprising:
providing or obtaining a suction system that includes a vacuum source, a suction regulator device, and a suction powered device fluidly connected to the suction regulator device via a suction line, the suction regulator device comprising:
a housing;
a first port of the housing connected to the vacuum source;
a second port of the housing exposing the suction line to ambient pressure;
a third port of the housing connected to the suction powered device via the suction line; and
a valve situated within the housing and switchable between an open configuration and a closed configuration, wherein in the open configuration the third port is fluidly connected the first port to thereby subject the suction line and the suction powered device to a pressure of the vacuum source, and wherein in the closed configuration the third port is fluidly connected to the second port to thereby subject the suction line and the suction powered device to ambient pressure;
delivering a tip of the suction powered device to a suction site in a patient;
maintaining the valve in the open configuration for a first predetermined time period with the vacuum source activated and the tip of the suction powered device at the suction site so as to draw both liquid bone matter and solid bone matter into the suction line through the tip; and
maintaining the valve in the closed configuration for a second predetermined time period with the vacuum source activated and the tip of the suction powered device at the suction site such that at least some of the liquid bone matter returns to the suction site through the tip and at least some of the solid bone matter remains in the suction line.

18. The method of claim 17, wherein the first predetermined time period ranges from about two seconds to about four seconds.

19. The method of claim 17, wherein the second predetermined time period ranges from about half a second to about two seconds.

20. The method of claim 17, wherein the suction system further comprises a controller situated within the housing for repeatedly switching the valve between the open configuration to the closed configuration with the vacuum source activated and the tip of the suction powered device at the suction site.
